(19) United States
(12) Patent Application Publication
(10) Pub. No.: US 2008/0319535 A1
(43) Pub. Date: Dec. 25, 2008

(54) Vascular Stent and Method of Making Vascular Stent

(75) Inventors: Michael Craven, Santa Rosa, CA (US); Michael Krivoruchko, Forestville, CA (US); Gianfranco Pelligrini, Santa Rosa, CA (US); Matthew Baldwin, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/767,826

(22) Filed: Jun. 25, 2007

(51) Int. Cl. A61F 2/06

(52) U.S. Cl. 623/1.22

(57) ABSTRACT

A stent and method of forming a stent include a wire bent into a waveform spirally wrapped into a hollow cylindrical shape. The waveform includes a first end portion, a middle portion, and a second end portion. The middle portion of the waveform includes a first amplitude and a first period. The first end portion of the waveform includes a first plurality of amplitudes and a first plurality of periods, wherein the first plurality of amplitudes decrease from adjacent the middle portion to a first end of the wire and first plurality of frequencies increase from adjacent the middle portion to the first end of the wire. The waveform may also include a second end portion with a second plurality of amplitudes and a second plurality of periods, wherein the second plurality of amplitudes decrease from adjacent the middle portion to a second end of the wire and the second plurality of frequencies increase from adjacent the middle portion to the second end of the wire.

### FIELD OF THE INVENTION

The present invention relates generally to stents and methods of making stents, and more particularly, to helical stents.

### BACKGROUND OF THE INVENTION

Cardiovascular disease, including atherosclerosis, is the leading cause of death in the U.S. The medical community has developed a number of methods and devices for treating coronary heart disease, some of which are specifically designed to treat the complications resulting from atherosclerosis and other forms of coronary arterial narrowing.

One method for treating atherosclerosis and other forms of coronary narrowing is percutaneous transluminal coronary angioplasty, commonly referred to as “angioplasty” or “PTCA”. The objective in angioplasty is to enlarge the lumen of the affected coronary artery by radial hydraulic expansion. The procedure is accomplished by inflating a balloon within the narrowed lumen of the coronary artery. Radial expansion of the coronary artery occurs in several different dimensions, and is related to the nature of the plaque. Soft, fatty plaque deposits are flattened by the balloon, while hardened deposits are cracked and split to enlarge the lumen. The wall of the artery itself is also stretched when the balloon is inflated.

Unfortunately, while the affected artery can be enlarged, in some instances the vessel restenoses chronically, or closes down acutely, negating the positive effect of the angioplasty procedure. In the past, such restenosis has frequently necessitated repeat angioplasty or open heart surgery. While such restenosis does not occur in the majority of cases, it occurs frequently enough that such complications comprise a significant percentage of the overall failures of the angioplasty procedure.

To lessen the risk of restenosis, various devices have been proposed for mechanically keeping the affected vessel open after completion of the angioplasty procedure. Such endoprostheses (generally referred to as “stents”), are typically inserted into the vessel, positioned across the lesion or stenosis, and then expanded to keep the passageway clear. The stent overcomes the natural tendency of the vessel walls of some patients to restenoses, thus maintaining the patency of the vessel.

Stents are delivered to the lesion, or target area, by a catheter device. Typically, the stent is introduced to the patient in an unexpanded form, having the smallest diameter possible. The small diameter is necessary during insertion in order to properly traverse tortuous blood vessels. When the stent reaches the target area, the stent is expanded to engage the blood vessel walls, enlarging the inner circumference of the blood vessel, and securing to vessel wall. When the stent is positioned across the target area, it is expanded, causing the length of the stent to contract and the diameter to expand.

The stent may be expanded by a number of methods, including expansion of the stent using a balloon on a balloon catheter. The balloon is inserted into the unexpanded stent, either before insertion to the patient or after the stent has reached the target site. The balloon is inflated while inside the circumference of the stent, forcing the stent to expand and lodge within the blood vessel at the target site.

Stents are generally formed using any of a number of different methods. One group of stents are formed by winding a wire around a mandrel, welding or otherwise forming the stent to a desired configuration, and finally compressing the stent to an unexpanded diameter. Another group of stents are manufactured by machining tubing or solid stock material into bands, and then deforming the bands to a desired configuration. Another group of stents are formed by laser etching or chemical etching, which cuts or etches a tube to a desired shape. The stent is usually etched or cut in an unexpanded state.

Helically wound stents, such as those described in U.S. Pat. No. 4,886,062 to Wiktor, the contents of which are incorporated herein by reference, generally comprise a wire formed into a waveform, such as a sinusoid, that is then helically wrapped around a mandrel to provide a tubular or cylindrical structure. Helically wound stents, however, generally include ends that are not substantially perpendicular to the longitudinal axis of the stent. In other words, due to the helical winding of the wire, a portion of each end of the stent extend further longitudinally than the remainder of each end of the stent, as shown in FIG. 2 of the Wiktor patent.

In some helically wound stents, such as those described in U.S. Pat. No. 5,314,472 to Fontaine, end portions of the wire have a reduced amplitude waveform as compared to the waveforms in the middle of the wire. Wrapping such a wire around a mandrel to form a stent results in a stent with ends that may be generally perpendicular to the longitudinal axis of the stent. However, due to the reduced amplitude at the ends of the wire, a greater force is required to expand the ends of the stent.

### BRIEF SUMMARY OF THE INVENTION

The present disclosure is directed to a stent and a method of making a stent. The stent is formed by bending a wire into a waveform. The waveform includes a first end portion, a middle portion, and a second end portion. The middle portion of the waveform includes a first amplitude and a first period. The first end portion of the waveform includes a first plurality of amplitudes and a first plurality of periods, wherein the first plurality of amplitudes decrease from adjacent the middle portion to a first end of the wire and first plurality of frequencies increase from adjacent the middle portion to the first end of the wire. The waveform may also include a second end portion with a second plurality of amplitudes and a second plurality of periods, wherein the second plurality of amplitudes decrease from adjacent the middle portion to a second end of the wire and the second plurality of frequencies increase from adjacent the middle portion to the second end of the wire. The waveform is spirally wound around a mandrel to form a hollow cylindrical shape of a stent.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a wire bent into a waveform for use in making a stent in accordance with an embodiment of the present invention.

FIG. 2 illustrates a detailed view of a portion of the waveform of FIG. 1.

FIG. 3 illustrates the waveform of FIG. 1 after it has been wrapped around a mandrel and is cut to lay flat for illustrative purposes.

FIG. 4 illustrates the waveform of FIG. 1 being wrapped around a mandrel.

### DETAILED DESCRIPTION OF THE INVENTION

Specific embodiments of the present invention are now described with reference to the figures, where like reference numbers indicate identical or functionally similar elements.

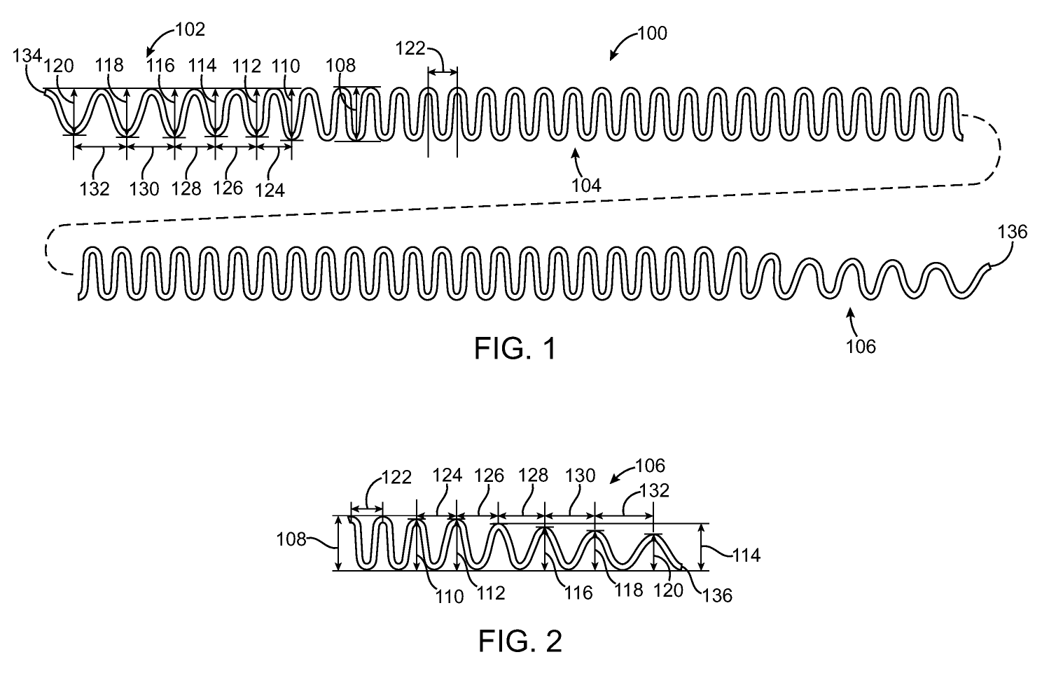

FIG. 1 shows a wire or filament 100 formed into a planar waveform. The terms “filament” and “wire” as used herein mean any elongated filament or group of filaments. The filament or wire may be made of any material, such as titanium, tantalum, gold, copper or copper alloys, combinations of these materials, or any other biologically compatible low shape-memory material. Further, several distinct filaments or wires may be attached together by any conventional means such as butt-welding in order to form a continuous filament or wire. Wire 100 includes a first end portion 102, a second end portion 106, and a middle portion 104 disposed between the first and second end portions 102,106. In the embodiment illustrated in FIG. 1, the waveform for middle portion 104 is substantially a sinusoid having amplitude 108 and a period 122. However, one of ordinary skill in the art would recognize that the waveform need not be a sinusoid, but can be any generally repeating pattern.

The waveform for first end portion 102 of wire 100 is also generally a sinusoid. However, the amplitude and period of the waveform of first end portion 102 varies as it extends from middle portion 104 to end 134 of wire 100. In particular, the period increases for each wave extending from middle portion 104 to end 134. Thus, period 124 is larger than period 122, period 126 is larger than period 124, period 128 is larger than period 126, period 130 is larger than period 128, and period 132 is larger than period 130, as illustrated in FIG. 1. The same pattern is repeated for second end portion 106, as illustrated in FIG. 2. As the period increases from middle portion 104 to end 134, the amplitude decreases. Thus, amplitude 110 is smaller than amplitude 108 of middle portion 104, amplitude 112 is smaller than amplitude 110, amplitude 114 is smaller than amplitude 112, amplitude 116 is smaller than amplitude 114, amplitude 118 is smaller than amplitude 116, and amplitude 120 is smaller than amplitude 118, as illustrated in FIG. 1. The same pattern is repeated for second end portion 106, as illustrated in FIG. 2.

One of ordinary skill in the art would recognize that each wave of first and second end portions 102 and 106 need not decrease in amplitude and increase in period. Some waves in first and second end portions 102 and 106 may be equal to adjacent waves in amplitude or period. Further, only one end portion may have decreasing amplitudes and increasing periods, and first and second end portions 102 and 106 need not be identical.

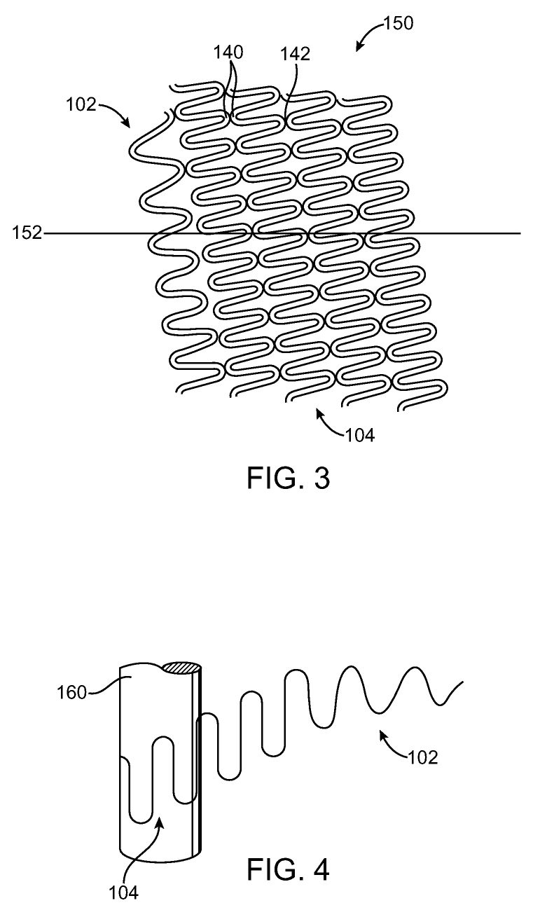

FIG. 4 shows a method of forming a stent 150 in accordance with an embodiment of the present invention by wrapping wire 100 around a mandrel 160. FIG. 3 illustrates stent 150 after it has been wrapped around mandrel 160. Stent 150 of FIG. 3 has been illustrated as if were cut longitudinally parallel to longitudinal or cylindrical axis 152 and laid flat. The circumference of the mandrel may be selected such that adjacent bends 140 of the waveform face each other, as illustrated in FIG. 3. Welds 142 may connect certain adjacent bends 142 together, as also illustrated in FIG. 3.

While several embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Further, it will be apparent to persons skilled in the relevant art that different features of the various embodiments may be combined with features of other embodiments without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A stent comprising:
a wire bent into a waveform spirally wrapped into a hollow cylindrical shape, the waveform including a first end portion, a middle portion, and a second end portion;
the middle portion of the waveform including a first amplitude and a first period;
the first end portion of the waveform including a second amplitude and a second period, wherein the second amplitude is smaller than the first amplitude and the second period is larger than the first period.

2. The stent of claim 1, wherein the entire middle portion of the waveform has the first amplitude and the first period.

3. The stent of claim 1, wherein the first end portion includes a plurality of amplitudes and frequencies, wherein the plurality of amplitudes are each smaller than the first amplitude and the plurality of frequencies are each larger than the first frequency.

4. The stent of claim 4, wherein the plurality of amplitudes in the first end portion decrease and the plurality of frequencies in the first end portion increase from adjacent the middle portion to a first end of the waveform.

5. The stent of claim 1, wherein the second end portion includes a third amplitude and a third period that are equal to the second amplitude and second period, respectively.

6. The stent of claim 1, wherein the second end portion includes a plurality of amplitudes and frequencies, wherein the plurality of amplitudes are each smaller than the first amplitude and the plurality of frequencies are each larger than the first frequency.

7. A stent comprising:
a wire bent into a waveform spirally wrapped into a hollow cylindrical shape, the waveform including a first end portion, a middle portion, and a second end portion;
the middle portion of the waveform including a first amplitude and a first period;
the first end portion of the waveform including a first plurality of amplitudes and a first plurality of periods, wherein the first plurality of amplitudes decrease from adjacent the middle portion to a first end of the wire and the first plurality of frequencies increase from adjacent the middle portion to the first end of the wire; and
the second end portion including a second plurality of amplitudes and a second plurality of frequencies, wherein the second plurality of amplitudes decrease from adjacent the middle portion to a second end of the wire and the second plurality of frequencies increase from adjacent the middle portion to the second end of the wire.

8. A method of forming a stent comprising the steps of:
bending a wire into a waveform, the waveform including a middle portion including a first amplitude and a first period, and a first end portion including a first plurality of amplitudes and a first plurality of periods, wherein the first plurality of amplitudes are less than the first period and the first plurality of periods are larger than the first period; and
spirally winding the wire into hollow cylindrical shape.

9. The method of claim 8, wherein the waveform further includes a second end portion having a second plurality of amplitudes and second plurality of periods, wherein the second plurality of amplitudes are smaller than the first amplitude and the second plurality of periods are larger than the first amplitude.

10. The method of claim 9, wherein the second plurality of amplitudes decrease from adjacent the middle portion to a second end of the wire.

11. The method of claim 9, wherein the second plurality of periods increase from adjacent the middle portion to a second end of the wire.

12. The method of claim 11, wherein the second plurality of amplitudes decrease from adjacent the middle portion to the second end of the wire.

13. The method of claim 8, wherein the first plurality of amplitudes decrease from adjacent the middle portion to a first end of the wire.

14. The method of claim 8, wherein the device of claim 9, wherein the first plurality of periods increase from adjacent the middle portion to a first end of the wire.

15. The method of claim 14, wherein the first plurality of amplitudes decrease from adjacent the middle portion to the first end of the wire.

16. The method of claim 15, wherein the waveform further includes a second end portion having a second plurality of amplitudes and second plurality of periods, wherein the second plurality of amplitudes are smaller than the first amplitude and the second plurality of periods are larger than the first amplitude.

17. The method of claim 16, wherein the second plurality of amplitudes decrease from adjacent the middle portion to a second end of the wire and the second plurality of periods increase from adjacent the middle portion to the second end of the wire.
